(19) United States
(12) Patent Application Publication
(10) Pub. No.: US 2023/0380750 A1
(43) Pub. Date: Nov. 30, 2023

(54) SYSTEMS AND METHODS OF ESTIMATING HEARING THRESHOLDS USING AUDITORY BRAINSTEM RESPONSES

(71) Applicant: University of Washington, Seattle, WA (US)

(72) Inventors: Erik Petersen, Seattle, WA (US); Yi Shen, Seattle, WA (US)

(21) Appl. No.: 18/324,004

(22) Filed: May 25, 2023

Related U.S. Application Data
(60) Provisional application No. 63/345,798, filed on May 25, 2022.

(51) Int. Cl. A61B 5/38; A61B 5/12; A61B 5/372; A61B 5/00

(52) U.S. Cl. CPC A61B 5/38; A61B 5/125; A61B 5/372; A61B 5/7246; A61B 2503/04

(57) ABSTRACT

Embodiments of the present disclosure provide methods, systems and non-transitory computer readable media of estimating hearing thresholds using auditory brainstem responses (ABR). An example method includes: presenting at least one stimulus to a subject; receiving first ABR signals responsive to the at least one stimulus; fitting a model to at least the first ABR signals to provide a fitted model; generating, using the fitted model, predicted hearing thresholds across a range of frequencies and uncertainty associated with the hearing thresholds; determining a next stimulus based, at least in part, on the uncertainty associated with the hearing thresholds; and presenting the next stimulus to the subject.

### RELATED APPLICATIONS

This application claims priority to U.S. Provisional Application 63/345,798, filed May 25, 2022, which application is hereby incorporated by reference, in its entirety, for any purpose.

### STATEMENT OF FEDERALLY SPONSORED RESEARCH AND DEVELOPMENT

This invention was made with government support under Grant No. R01DC017988 and Grant No. 2 T32 DC 5361-21, both awarded by the National Institutes of Health (NIH). The government has certain rights in the invention.

### TECHNICAL FIELD

Examples described herein relate to estimating hearing thresholds using auditory brainstem responses (ABR). Examples of methods herein are described to predict hearing thresholds using a fitted model using ABR signals responsive to stimuli.

### BACKGROUND

Infants with hearing loss are at greater risk of abnormal cognitive development and slower speech acquisition compared to their normal hearing peers. Medical care for hearing loss and early detection, diagnosis, and treatment of hearing loss through the prescription of hearing aids or cochlear implants are essential components of medical care during the first months of life. Since infants tend to have difficulty in responding to typical behavioral procedures, clinicians often rely on electrophysiological methods using auditory evoked potentials, such as ABR, to determine hearing thresholds. Infants who have been identified as hearing-impaired during a screening process are referred to undergo a comprehensive diagnosis, which often involves ABR testing.

The ABR and other electrophysiological testing methods have been used in hearing health applications for decades. There have been continued efforts in developing methodologies for collecting and analyzing evoked potentials. For example, speech-based frequency-following response, binaural interaction component, and acoustic change complex have been explored to provide insights into neural encoding of sound during development. Certain techniques, such as interleaving frequencies and adaptively varying the number of averages, have been developed to improve efficiency of ABR audiometry.

ABR testing is time consuming because each stimulus is repeated many times and averaged to obtain an acceptable signal to noise ratio, and any measurements that coincide with excess movement or vocalizations of the infant may be rejected. Therefore, few frequencies are tested during a single session, leading to either an incomplete diagnosis or additional appointments at the clinic.

### SUMMARY

Examples described herein are directed towards systems, methods and computer readable media for estimating hearing thresholds based on ABR response signals. An example method includes presenting at least one stimulus to a subject; receiving first ABR signals responsive to the at least one stimulus; fitting a model to at least the first ABR signals to provide a fitted model; generating, using the fitted model, predicted hearing thresholds across a range of frequencies and uncertainty associated with the hearing thresholds; determining a next stimulus based, at least in part, on the uncertainty associated with the hearing thresholds; and presenting the next stimulus to the subject.

In some examples, the method may further include performing at least one iterative operation, including: receiving a second ABR signal responsive to the next stimulus; fitting the previously fitted model to at least the second ABR signal to provide a second fitted model; generating, using the second fitted model, second predicted hearing thresholds across a range of frequencies and uncertainty associated with the second hearing thresholds; determining another next stimulus based, at least in part, on the uncertainty associated with the second hearing thresholds; and presenting the other next stimulus to the subject.

In some examples, presenting the at least one stimulus includes providing at least one audio signal corresponding to the at least one stimulus to at least one sound reproducing device in proximity to a subject; and the first ABR signals include voltages provided by a sensor on the subject.

In some examples, generating the first predicted hearing thresholds includes: predicting amplitudes of ABR signals responsive to stimuli across the range of frequencies and levels using the fitted model; comparing the predicted amplitudes of the ABR signals with an amplitude of noise of the ABR signals; and generating the predicted hearing thresholds based on the comparison. In some examples, the method may further include calculating peak-to-peak amplitudes of the predicted ABR signals, amplitudes of individual peaks in the predicted ABR signals or latencies of individual peaks in the predicted ABR signals, generated using the fitted model.

In some examples, the fitted model may be used to predict ABR amplitude as a function of time, frequency and sound pressure level of the stimuli. In some examples, the model may be a Gaussian Process model. In some examples, fitting the model may include calculating covariance between pairs of variables across a sound pressure level using a linear kernel, calculating covariance between pairs of variables across time using a squared exponential kernel, calculating covariance between pairs of variables across base-two logarithm of frequency using a squared exponential kernel, and/or calculating covariance between pairs of variables across a level using a covariance kernel that is a product of a squared exponential kernel and a linear kernel.

In some examples, determining the next stimulus includes selecting at least one of a frequency or a sound pressure level to reduce uncertainty associated with the hearing thresholds. In some examples, the uncertainty associated with the hearing thresholds may be based on variance of amplitudes of one or more ABR waveforms generated using the fitted model.

An example method includes an ABR testing device and a computer coupled to the ABR testing device. The computer may include a processor and a non-transitory computer readable medium storing computer-executable instructions which, when executed, cause the processor to perform operations including: causing the ABR testing device to present at least one stimulus to a subject; receiving first ABR signals from the ABR testing device responsive to the at least one stimulus; fitting a model to at least the first ABR signals to provide a fitted model; generating, using the fitted model, predicted hearing thresholds across a range of frequencies and uncertainty associated with the hearing thresholds; determining a next stimulus based, at least in part, on the uncertainty associated with the hearing thresholds; and presenting the next stimulus to the subject.

In some examples, the ABR testing device may include the computer.

In some examples, the ABR testing device may include stimuli presentation circuitry configured to provide at least one audio signal corresponding to the at least one stimulus to a sound conduction device on the subject that is coupled to the ABR testing device.

In some examples, the ABR testing device may include data acquisition circuitry coupled to at least one sensor in proximity to the subject. The data acquisition circuitry may provide the first ABR signals extracted from ongoing electrical activity of the subject by the sensor. In some examples, the at least one sensor may be an electrode on the subject, and the at least one sensor may measure the first ABR signals responsive to the at least one auditory evoked potential.

An example non-transitory computer readable medium encoded with instructions which, when executed, cause a system to perform operations including: presenting at least one stimulus to a subject; receiving first ABR signals responsive to the at least one stimulus; fitting a model to at least the first ABR signals to provide a fitted model; generating, using the fitted model, predicted hearing thresholds across a range of frequencies and uncertainty associated with the hearing thresholds; determining a next stimulus based, at least in part, on the uncertainty associated with the hearing thresholds; and presenting the next stimulus to the subject.

In some examples, the operations of the non-transitory computer readable medium may further include performing at least one iterative operation including: receiving a second ABR signal responsive to the next stimulus; fitting the previously fitted model to at least the second ABR signal to provide a second fitted model; generating, using the second fitted model, second predicted hearing thresholds across a range of frequencies and uncertainty associated with the second hearing thresholds; determining another next stimulus based, at least in part, on the uncertainty associated with the second hearing thresholds; and presenting the next stimulus to the subject.

In some examples, generating the first predicted hearing thresholds may include predicting amplitudes of ABR signals responsive to stimuli across the range of frequencies using the fitted model; comparing the predicted amplitudes of the ABR signals with an amplitude of noise of the ABR signals; and generating the first predicted hearing thresholds based on the comparison.

In some examples, determining the next stimulus may include selecting at least one of a frequency or a sound pressure level to reduce the uncertainty associated with the hearing thresholds. In some examples, the uncertainty associated with the hearing thresholds is based on variance of amplitudes of one or more ABR waveforms generated using the fitted model.

### BRIEF DESCRIPTION OF THE DRAWINGS

FIG. 1 is a schematic illustration of a system of estimating hearing thresholds using ABR signals in accordance with examples described herein.

FIG. 2 is a schematic illustration of a system of estimating hearing thresholds using ABR signals in accordance with examples described herein.

FIG. 3 is a schematic illustration of a flowchart of estimating hearing thresholds using ABR signals in accordance with examples described herein.

FIG. 4 shows an equation representing a linear kernel of a Gaussian Process (GP) model in accordance with examples described herein.

FIG. 5 shows an equation representing a squared exponential kernel of a GP model in accordance with examples described herein.

FIGS. 6-9 are diagrams of ABR signals and threshold estimates based on a set of stimuli in accordance with examples described herein.

FIG. 10 shows example ABR signals data sets of a system of estimating hearing thresholds using ABR signals in accordance with examples described herein.

### DETAILED DESCRIPTION

The following description of certain embodiments is merely exemplary in nature and is in no way intended to limit the scope of the disclosure or its applications or uses. In the following detailed description of embodiments of the present systems, devices, and methods, reference is made to the accompanying drawings which form a part hereof, and which are shown by way of illustration specific to embodiments in which the described systems, methods and computer readable media may be practiced. In some instances, well-known circuits, control signals, timing protocols, and/or software operations have not been shown in detail in order to avoid unnecessarily obscuring the described embodiments. It is to be understood that other embodiments may be utilized and that structural and logical changes may be made, without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense for the appended claims.

Systems, methods and computer readable media for estimating hearing thresholds with fewer presentation stimuli using a fitted model may be advantageous as they may reasonably estimate hearing thresholds for audio stimuli without presenting numerous stimuli for predicting the hearing thresholds. Thus, examples of such systems, methods and/or computer readable media may improve accuracy of the hearing thresholds estimation for subjects with difficulty in going through many stimuli (e.g., babies, young children, animals, etc.) while reducing the burden to such subjects. Thus examples described herein may allow medical professionals and/or researchers to provide appropriate hearing aids to remedy the subjects with too high too low hearing thresholds across certain frequencies based on the improved estimation of the hearing thresholds.

Examples of systems, methods and computer readable media for estimating hearing thresholds with fewer presentation stimuli using a fitted model may be implemented into a comprehensive diagnostic protocol for use in the clinical environment. In order to improve the time efficiency, Bayesian adaptive estimation may be integrated in ABR audiometry as described herein. The ABR-based adaptive estimation may be combined with emerging interests in computational (e.g. artificial intelligence/machine learning) audiology. Examples of technology in this disclosure may improve ABR audiometry that is used for hearing aid/cochlear implant candidacy determination.

Advantageously, examples of systems, methods and/or computer readable media described herein may utilize model fitting based on ABR signals responsive to a reduced number of presentation stimuli. Examples of ABR estimation utilizing such systems, methods and/or computer readable media may be implemented in ABR testing systems including desktop or laptop computers, a smart device such as a smartphone or a wearable device coupled to one or more ABR testing devices, or standalone portable ABR testing devices. In addition, examples of systems, methods and/or computer readable media described herein may be implemented in portable ABR testing devices, or portable ABR testing devices with portable smart devices that may act as edge devices, such as smartphones and wearables. While various advantages of example systems, methods and/or computer readable media have been described, it is to be understood that not all examples of the described technology may have all, or even any, of the described advantages.

Generally, ABR signals described herein refer to a type of auditory evoked potential. Generally, ABR signals may refer to voltage, current, or other parameter traces over time received from a subject (e.g., a brain of a subject) responsive to an auditory stimulus presented to the subject.

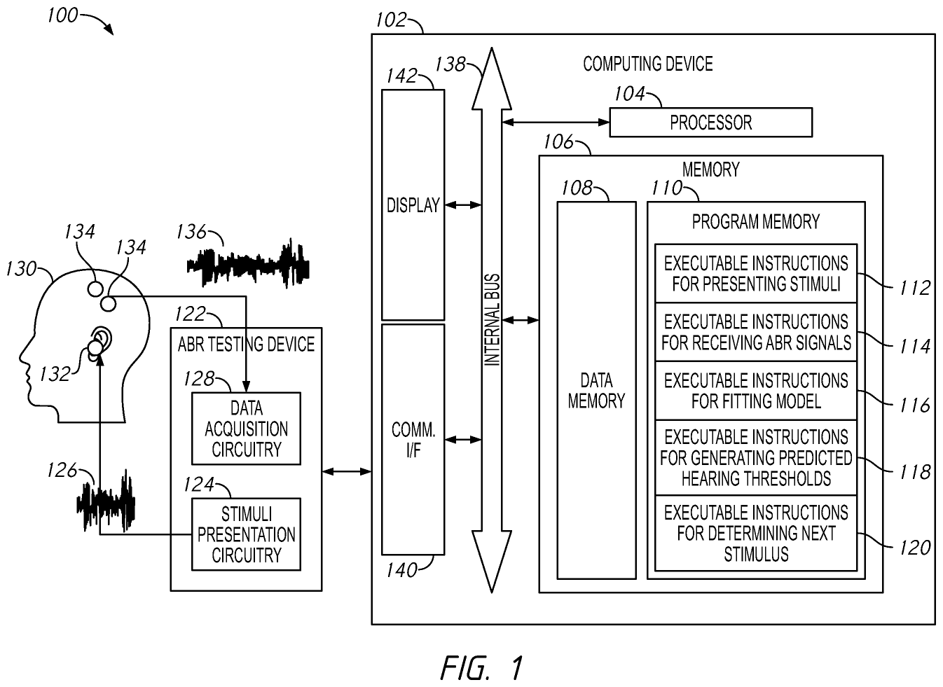

FIG. 1 is a schematic illustration of a system 100 of estimating hearing thresholds using ABR signals in accordance with examples described herein. The system 100 includes a computing device 102 and an ABR testing device 122 coupled to the computing device 102. The computing device 102 includes components, such as a processor 104, memory 106, communication interface 140, display 142, and internal bus 138. The memory 106 may include data memory 108 and program memory 110. The program memory 110 may be a non-transitory computer readable medium storing computer-executable instructions, including executable instructions for presenting stimuli 112, executable instructions for receiving ABR signals 114, executable instructions for fitting model 116, executable instructions for generating predicted hearing thresholds 118, and/or executable instructions for determining next stimulus 120. The ABR testing device 122 may include stimuli presentation circuitry 124, and data acquisition circuitry 128. The stimuli presentation circuitry 124 may provide an audio signal 126 to a subject 130 through one or more sound conduction devices 132 coupled to the stimuli presentation circuitry 124 and further attached to the subject 130. The data acquisition circuitry 128 may be coupled to one or more sensors 134 that are placed in proximity to the subject 130. In some examples, the one or more sensors 134 may be attached to the subject 130. The data acquisition circuitry 128 may receive one or more ABR signals 136 from the one or more sensors 134. In some examples, the first ABR signals may include voltages provided by the one or more sensors 134 on the subject.

The components of FIG. 1 are intended to be exemplary only. Additional, fewer, and/or different components may be used in other examples. In some examples, the computing device 102 may be wholly and/or partially integrated into ABR testing device 122. In some examples, the computing device 102 may be remote from ABR testing device 122.

Examples of systems described herein may include a computing device, such as a computing device 102. A computing device, such as the computing device 102 may be implemented using a desktop or laptop computer, a smart device such as a smartphone or a wearable device, a workstation or any computing device that may have computational functionality that may be configured to be coupled to (e.g., be in communication with) one or more ABR testing devices, such as the ABR testing device 122.

Examples of computing devices described herein may generally include one or more processors, such as a processor 104 of FIG. 1. Processors, such as processor 104, may be implemented using one or more central processing units (CPUs), graphics processing units (GPUs), field programmable gate arrays (FPGAs), application specific integrated circuits (ASICs), controllers, microcontrollers, or other circuitry. Processors described herein may be used to implement software and/or firmware systems. For example, the processor 104 may be used to execute one or more executable instructions encoded on computer readable media (e.g., memory). While a single processor 104 is shown in FIG. 1, it is to be understood that any number of processors may be used, and multiple processors may be in communication with each other to perform functions of a processor described herein. The processor 104 may be coupled to (e.g., in communication with) memory 106, communication interface 140, and/or one or more displays 142 of FIG. 1 via an internal bus 138.

Examples of computing devices described herein may include memory, such as memory 106 of FIG. 1. Generally, any number or kind of memory may be used including read-only memory (ROM), random access memory (RAM), flash memory, one or more solid state drives, one or more hard disks, one or more secure digital (SD) cards, or other computer readable media. The memory 106 may include program memory 110 and data memory 108. In some examples, the program memory 110 and the data memory 108 may be implemented as separate segments of the memory 106 as one or more integrated memory devices. In some examples, the program memory 110 and the data memory 108 may be implemented as separate memory devices of the same kind or different kinds. In some examples, any of the program memory 110 and/or the data memory 108 may be fixed in the computing device 102. In some examples, any of the program memory 110 and/or the data memory 108 may be attachable to and detachable from the computing device 102. The program memory 110 may be implemented as a non-transitory computer readable medium storing computer-executable instructions, such as executable instructions for presenting stimuli 112, executable instructions for receiving ABR signals 114, executable instructions for fitting model 116, executable instructions for generating predicted hearing thresholds 118, and executable instructions for determining next stimulus 120.

The data memory 108 described herein may store data. In some examples, the data to be stored in the data memory 108 may include, for example, data for performing instructions encoded in the program memory 110, including a set of ABR signals to be used for fitting model, parameters to represent fitted model, predicted hearing thresholds and uncertainty associated with the hearing thresholds. In some examples, the data stored in the data memory 108 may include data to be exchanged with external devices, such as the ABR testing device 122. For example, the data to be exchanged may include information about a stimulus to be presented. In some examples, the information about a stimulus may be an audio signal to be presented as a stimulus to be provided from the computing device 102 to the ABR testing device 122. In some examples, the information about a stimulus may be a combination of parameters which may cause the ABR testing device 122 to present the stimulus as the audio signal, or the audio signal itself. In some examples, the stimulus may be either an audio signal selected among a set of audio signals stored in the data memory 108 or tentatively generated, based on the uncertainty associated with the hearing thresholds across parameters, such as frequencies and/or time temporarily stored in the data memory 108. In some examples, the data to be exchanged may include a set of ABR signals received from the ABR testing device 122 responsive to the stimuli presented. While a single memory 106 is shown in FIG. 1, it is to be understood that any number of memory devices may be used, and the executable instructions and/or data may be distributed across multiple memories accessible to the processor 104.

Examples of computing devices described herein may include additional components. For example, the computing device 102 may include or be coupled to output devices. In some examples, the output devices may be one or more display(s), such as a display 142 of FIG. 1, and/or speakers. While FIG. 1 shows the display 142 integrated into the computing device 102, the display 142 or any output devices may be external devices coupled to the computing device 102. For example, the computing device 102 may include or be coupled to input devices. In some examples, the input device(s) may include keys, buttons, keyboards, mice, touchscreens, microphones, etc. The additional components in the computing device 102 may communicate with the processor 104 and/or the memory 106 of FIG. 1 via the internal bus 138. For example, the computing device 102 may further include an internal bus 138 that may provide communications between components included in the computing device 102, such as the processor 104, the memory 106, the communication interface 140 and the display 142. The computing device 102 may further include communication interface 140 (e.g., cellular antenna, Wi-Fi, network interface, Bluetooth interface) of FIG. 1 that may communicate wirelessly or via wire(s) such as universal serial bus (USB) cables, ether cables, Hi-definition multimedia interface (HDMI) cables, or other standardized or proprietary cables. The additional components and/or the ABR testing device 122 coupled to the computing device 102 may communicate with the computing device 102 via the communication interface 140. The communication interface 140 may receive and/or transmit communications between the computing device 102 and external devices, including the ABR testing device 122.

Examples of systems described herein may include an ABR testing device, such as the ABR testing device 122. The ABR testing device 122 may include stimuli presentation circuitry and data acquisition circuitry. The stimulation presentation circuitry may be stimuli presentation circuitry 124 of FIG. 1. The stimuli presentation circuitry 124 may receive either the information about a stimulus that may be a combination of parameters (e.g., one or more frequencies and levels) which may cause the ABR testing device 122 to present the stimulus as the audio signal, or the audio signal itself from the computing device 102. The audio signal accordingly may have one or more particular frequencies at one or more particular levels (e.g., sound level, which may be measured in decibels, dB). Responsive to the information about a stimulus, the stimuli presentation circuitry 124 may present the stimulus via one or more sound conduction devices 132 that may be coupled to the stimuli presentation circuitry 124 via wires (e.g., analog audio cables or USB cables) or wirelessly (e.g., Bluetooth interface). In some embodiments, the sound conductive devices 132 may be air conductive devices, such as one or more earphones or headphones to be attached to one or more corresponding ears of the subject 130 or speakers placed in proximity to the subject 130. In some embodiments, the sound conductive devices 132 may be bone conductive devices, such as one or more speakers, headphones or vibrators to be attached to a head of the subject 130. The data acquisition circuitry may be data acquisition circuitry 128 of FIG. 1. The data acquisition circuitry 128 may be coupled to one or more sensors 134 placed in proximity to the subject 130. In some examples, the one or more sensors 134 may be one or more electrodes. In some examples, the one or more sensors 134 may be attached to a head of the subject 130. In some examples, the data acquisition circuitry 128 may include one or more bio-amplifiers. In some examples, the one or more bio-amplifiers may be integrated within the ABR testing device 122. In some examples, the one or more bio-amplifiers may be integrated into the one or more sensors 134, such as electrodes. The data acquisition circuitry 128 may receive one or more ABR signals 136 from the one or more sensors 134.

A number of stimuli may be presented to a subject. During an initialization process, a small number (e.g., five to seven) of stimuli may be presented. Examples of systems and methods described herein may determine a next stimulus to present, which may be based on reducing uncertainty in an estimate of hearing thresholds. During an iteration process, a next stimulus may be presented. The processor 104 of the computing device 102 may execute the executable instructions for presenting stimuli 112 to cause the system 100 to determine and/or present one or more stimuli. For example, the computing device 102 may provide information about the one or more stimuli to the ABR testing device 122 through the communication interface 140. For example, an amplitude and/or a frequency of the next stimulus may be provided. The stimuli presentation circuitry 124 of the ABR testing device 122 may present the one or more stimuli by reproducing a number of audio signals 126 corresponding to the one or more stimuli through the sound conduction device 132. In some examples, the one or more stimuli may be presented through air conductive devices, such as one or more earphones to be fitted in the one or more corresponding ears of the subject 130, headphones on the one or more corresponding ears of the subject 130 or speakers in proximity to the subject 130. In some examples, the one or more stimuli may be presented by bone conductive devices, such as one or more speakers, headphones or vibrators to be attached to the head of the subject 130.

The processor 104 may execute the executable instructions for receiving ABR signals 114 to cause the system 100 to receive one or more ABR signals responsive to the one or more stimuli that have been reproduced. In some examples, the data acquisition circuitry 128 of the ABR testing device 122 may provide the one or more ABR signals. In some examples, the one or more ABR signals may be auditory evoked potentials in waveforms for one or more corresponding synapses that may occur at different latencies. In some examples, the data acquisition circuitry 128 may obtain at least one auditory evoked potential extracted from ongoing electrical activity of the subject 130 by a sensor 134 of the one or more sensors 134. The ABR testing device 122 may provide the one or more ABR signals to the computing device 102. The communication interface 140 of the computing device 102 may receive the one or more ABR signals at the communication interface 140, and store the one or more ABR signals in the data memory 108.

The processor 104 may perform the executable instructions for fitting model 116 to cause the system 100 to fit a model to at least the received ABR signals to provide a fitted model. Generally, the process of fitting the model may include generating one or more predicted ABR waveforms. The processor 104, in accordance with the executable instructions for fitting model 116, may accordingly utilize received ABR signals that are responsive to particular stimulus frequencies and/or levels to generate predicted ABR signals that predict the subject's response to other stimulus frequencies and/or levels. Note that the model may be fitted across time, frequency, and level (e.g., sound level). For example, fitting the model may generate predicted ABR signals across time that are associated with different stimulus frequencies and/or levels. In this manner, a parameter space of time, frequency, and level may be populated with received ABR signals and predicted ABR signals. Fitting the model also generates a variance associated with the amplitude of the predicted ABR signals (e.g., the predicted ABR waveforms). This variance may reflect an uncertainty of the model (e.g., an uncertainty of the predicted ABR signals). This uncertainty, as described herein, may be utilized by systems to estimate an uncertainty associated with hearing thresholds.

In some examples, the model may be a GP model. In some examples, the fitted model that is used to predict ABR signals may be represented as a function of time, frequency and sound pressure level of the stimuli. Predicted ABR signals may be represented as a particular amplitude over time. The predicted ABR signal, occurring over a particular time, may be associated with a stimulus level and frequency. Note that the associated stimulus level and frequency may not have been presented to the subject. Rather, the predicted ABR signal is an estimate of the ABR signal that would be received if the subject was presented with a stimulus at a particular level or frequency. In some examples, the model may be fitted across two or more parameters. In some examples, the two or more parameters may include any of time, frequency, and level. In some examples, the model may be fitted across time, frequency, and level simultaneously.

In some examples, in order to fit the model (e.g. in order to generate one or more predicted ABR signals), the processor 104 may calculate covariance between pairs of variables. In some examples, the calculation may include calculating covariance between pairs of variables across sound pressure level using a linear kernel. In some examples, the calculation may include calculating covariance between pairs of variables across time using a squared exponential kernel. In some examples, the calculation may include calculating covariance between pairs of variables across base-two logarithm of frequency using a squared exponential kernel. In some examples, the calculation may include calculating covariance between pairs of variables across sound pressure levels using a covariance kernel that is a product of a squared exponential kernel and a linear kernel. The fitted model may accordingly include estimated ABR signals responsive to stimuli at frequencies and/or levels that had not yet been presented to the subject. In this manner, the fitted model may include estimated ABR signals that are fitted to ABR signals which have been received from the subject. In this manner, the ABR signals which have been received responsive to stimuli presented to the subject may be used by fit a model including creating estimates of additional ABR signals responsive to frequency and/or amplitude levels not yet presented to the subject.

Note that examples of Gaussian process models may be used described herein. Examples of Gaussian process models include non-parametric, statistical models. Examples of Gaussian process models may utilize minimal a priori knowledge of an expected shape of a response. Gaussian process models described herein may be different than a parametric model, which may typically model a known or expected shape, such as a linear, polynomial, and/or sine wave shape. Accordingly, a Gaussian process model may be used, based on ABR signals collected, to predict ABR signals for a subject responsive to frequencies and/or levels of stimuli that have not been provided. This fitting of the Gaussian process model to the known received ABR signals may include in some examples modelling data as a Gaussian process. Examples may include modelling how amplitude covaries based on frequency—e.g., frequencies that are closer may generally have a greater covariance. In this manner, a covariance kernel may be modelled in accordance with techniques described herein. ABR amplitude may be modelled across time, frequency, and level (e.g., amplitude) in examples described herein.

The processor 104 may further perform executable instructions for generating predicted hearing thresholds 118 to cause the system 100 to generate, using the fitted model, predicted hearing thresholds 118 across a range of frequencies. The fitted model may also be used to generate uncertainty associated with the hearing thresholds. In some examples, the processor 104 may calculate peak-to-peak amplitudes of the received ABR signals and/or predicted ABR signals generated using the fitted model. Accordingly, the processor 104, in accordance with the executable instructions for generating predicted hearing thresholds 118 may represent the ABR signals (either received and/or predicted) associated with a particular stimulus level using a metric, such as a peak-to-peak amplitude of the signal.

The processor 104, in accordance with the executable instructions for generating predicted hearing thresholds 118 may calculate or otherwise generate hearing thresholds at multiple frequencies. The hearing thresholds may be based on the metrics used to represent the ABR signals (e.g., peak-to-peak amplitudes). In some examples, a hearing threshold may be generated based on one or more statistical criteria for the metric (e.g., a sound level at which the peak-to-peak amplitude of the ABR signal(s) exceed a noise floor). In some examples, the statistical criterion may be the peak-to-peak amplitude of the ABR signals exceeds a predetermined tolerance (e.g., a noise floor). While a peak-to-peak metric is described, other metrics may be used to represent ABR signals and determine a hearing threshold in other examples. The tolerance may be set as a constant or a set based on the statistics that represents a relative strength of the brainstem response to a measurement noise. In some examples, the processor 104 may predict amplitudes of ABR signals responsive to stimuli across the range of frequencies using the fitted model, compare the predicted amplitudes of the ABR signals with an amplitude of noise of the ABR signals, and generate predicted hearing thresholds based on the comparison. In some examples, a threshold is set as the level at which a metric of an amplitude of the predicted ABR waveform using the fitted model is above the amplitude of noise according to a set statistical criterion. In some examples, the processor 104 may calculate a variance of amplitude of the predicted ABR waveform using the fitted model, and generate the uncertainty associated with the hearing thresholds based on the variance of the amplitude of predicted ABR waveform.

The processor 104 may further perform executable instructions for determining next stimulus 120 to cause the system to determine a next stimulus. In some examples, the processor 104 may determine the next stimulus based, at least in part, on the uncertainty associated with the hearing thresholds. In some examples, the processor 104 may select at least one of a frequency or a sound pressure level to reduce uncertainty associated with the hearing thresholds. The processor 104 may further cause the system 100 to present the next stimulus to the subject 130. Uncertainty as referred to herein may include an estimated error associated with a predicted hearing threshold associated with a variance of amplitude of an ABR waveform. Uncertainty values may be obtained across frequency and amplitude levels of a fitted model described herein, and/or uncertainty values may be obtained associated with estimated hearing thresholds across frequencies described herein.

In some examples, the processor 104 may evaluate whether one or more termination criteria are met. Examples of termination criteria include having uncertainty in the predicted hearing threshold be less than a threshold amount of uncertainty over a frequency range (e.g., an audible frequency range). Examples of termination criteria include having a change in the uncertainty be less than a threshold amount of change between iterations. Examples of termination criteria may include a fixed number (e.g. 15) of stimuli. In some examples, the change in predicted threshold may be tracked for consecutive iterations, and the iterations may be terminated when the predicted threshold stops changing significantly from one iteration to another. For example, the iterations may be terminated when the difference between consecutive predicted thresholds becomes smaller than a predetermined value.

In some examples, the GP model uncertainty may be leveraged to derive a termination criterion. For example, the termination criterion may be based on the change in the variance of the predicted ABR waveform. In some examples, the termination may be initiated by human intervention. For example, an audiologist, a clinical technician, or a lab technician may monitor data acquisition and make a subjective determination on when to terminate the procedure based on observation of the data (e.g., on a visual display of a collected ABR signal or an interim estimate of hearing threshold). The user may provide an input (e.g., through a user interface of the ABR system). The input may be, for example, clicking or touching a button or other graphical element displayed on a display of the ABR system or other computing system in communication with the ABR system. Responsive to the user input, systems described herein may terminate iterations of model fitting.

Once a termination criteria is met, a final predicted hearing threshold may be identified (e.g., a last predicted hearing threshold generated in accordance with the executable instructions for generating predicted hearing thresholds 118) and may become the final hearing threshold. The final hearing threshold may be reported by the computing device 102 by, for example, being displayed on display 142 and/or transmitted to one or more other computing devices. The final hearing threshold may be viewed and/or perceived by one or more users, such as an audiologist. If the final hearing threshold is indicative of hearing loss in the subject, a user may take action based on the final hearing threshold. For example, the user may perform additional testing, fit the subject for a cochlear implant, fit hearing aids, and/or other intervention.

It should be understood that this and other arrangements and elements (machines, interfaces, function, orders, and groupings of functions, etc.) can be used in addition to or instead of those shown, and some elements may be omitted altogether. Various functions described herein as being performed by one or more components may be carried out by firmware, hardware, and/or software. For instance, and as described herein, various functions may be carried out by a processor executing instructions in one or more ABR testing devices.

In this manner, final predicted hearing thresholds described herein may be generated in an efficient manner. By selecting next stimulus levels and/or frequencies at levels where predicted ABR signals had a highest variance and/or other criteria impacting an uncertainty of a hearing threshold, examples described herein may reduce an overall number of stimulus that may be presented to a subject to generate final hearing thresholds. Instead of presenting stimuli at all represented frequencies and levels, a selection of stimuli are used that nonetheless are able to generate hearing thresholds with acceptable uncertainty. Examples of the systems and methods described herein select the particular stimuli to be used based on reducing overall uncertainty in the hearing thresholds. Because fewer stimuli may need to be presented to a subject, examples of systems and methods described herein may save significant time.

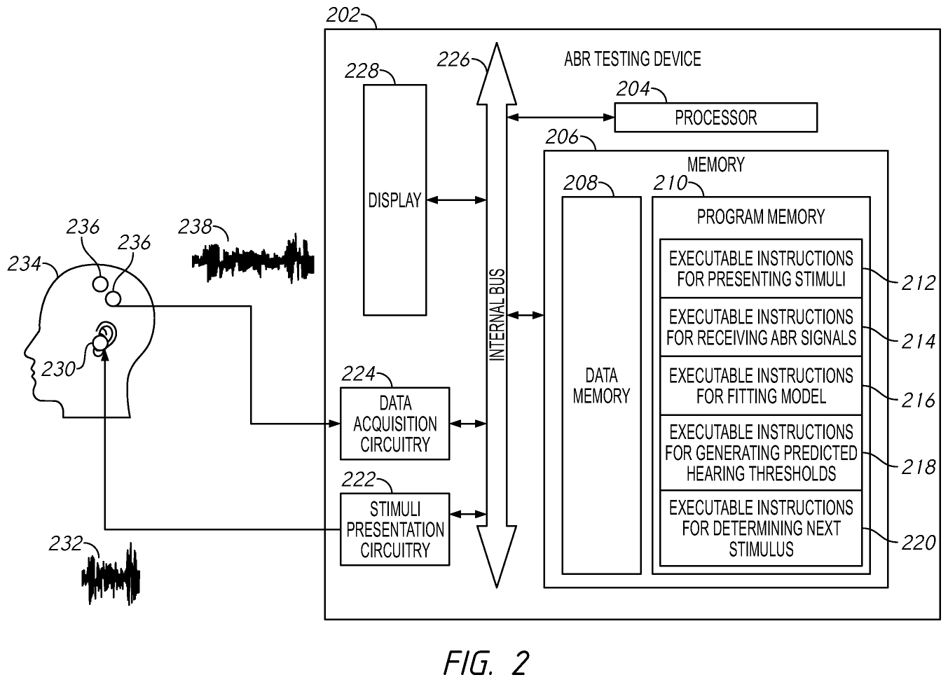

FIG. 2 is a schematic illustration of an ABR testing device 202 of estimating hearing thresholds using ABR signals in accordance with examples described herein. The ABR testing device 202 includes components, such as a processor 204, a memory 206, stimuli presentation circuitry 222, and data acquisition circuitry 224. The ABR testing device 202 may also include an internal bus 226 that may provide communications between components included in the ABR testing device 202, such as the processor 204, the memory 206, the stimuli presentation circuitry 222 and the data acquisition circuitry 224. The ABR testing device 202 may also include a display 228. The memory 206 may include data memory 208 and program memory 210. The program memory 210 may be a non-transitory computer readable medium storing computer-executable instructions, including executable instructions for presenting stimuli 212, executable instructions for receiving ABR signals 214, executable instructions for fitting model 216, executable instructions for generating predicted hearing thresholds 218, and executable instructions for determining next stimulus 220.

The ABR testing device 202 may be wholly and/or partially implemented using the computing device 102 of FIG. 1. For example, the memory 206 may be implemented using the memory 105 of FIG. 1. The executable instructions for presenting stimuli 212 may be implemented using the executable instructions for presenting stimuli 122 of FIG. 1, the executable instructions for receiving ABR signals may be implemented using the executable instructions for receiving ABR signals 114 of FIG. 1, the executable instructions for fitting model 216 may be implemented using executable instructions for fitting model 116 of FIG. 1, the executable instructions for generating predicted hearing thresholds 218 may be implemented using executable instructions for generating predicted hearing thresholds 118 of FIG. 1, and/or the executable instructions for determining next stimulus 220 may be implemented using the executable instructions for determining next stimulus 120 of FIG. 1.

The components of FIG. 2 are exemplary. Additional, fewer, and/or different components may be used in other examples.

The ABR testing device 202 may include stimuli presentation circuitry 222, and data acquisition circuitry 224. The stimuli presentation circuitry 222 may provide an audio signal 232 to a subject 234 through one or more sound conduction devices 230 coupled to the stimuli presentation circuitry 222 and further attached to the subject 234. The data acquisition circuitry 224 may be coupled to one or more sensors 236 that are placed in proximity to the subject 234. In some examples, the one or more sensors 236 may be attached to the subject 234. The data acquisition circuitry 224 may receive one or more ABR signals 238 from the one or more sensors 236.

In some examples, such instructions may directly control operations of the stimuli presentation circuitry 222 and the data acquisition circuitry 224. In some examples, the processor 204 of the ABR testing device 202 may perform the executable instructions for presenting stimuli 212 to cause the ABR testing device 202 to present one or more stimuli. For example, the processor 204 may provide information about the one or more stimuli to the stimuli presentation circuitry 222. The stimuli presentation circuitry 222 may present the one or more stimuli by reproducing a number of audio signals 232 corresponding to the one or more stimuli through the sound conduction device 230. In some examples, the one or more stimuli may be presented through air conductive devices, such as one or more earphones to be fitted in the one or more corresponding ears of the subject 234, headphones on the one or more corresponding ears of the subject 234 or speakers in proximity to the subject 234. In some examples, the one or more stimuli may be presented by bone conductive devices, such as one or more speakers, headphones or vibrators to be attached to the head of the subject 234. In some examples, the processor 204 may perform the executable instructions for receiving ABR signals 214 to cause the ABR testing device 202 to receive one or more ABR signals 238 responsive to the one or more stimuli that have been reproduced. In some examples, the data acquisition circuitry 224 of the ABR testing device 202 may provide the one or more ABR signals. In some examples, the one or more ABR signals may be auditory evoked potentials in waveforms for one or more corresponding synapses that may occur at different latencies. In some examples, the data acquisition circuitry 224 may obtain at least one auditory evoked potential extracted from ongoing electrical activity of the subject 234 by a sensor 236 of the one or more sensors 236, and the processor 204 may store the one or more ABR signals in the data memory 208.

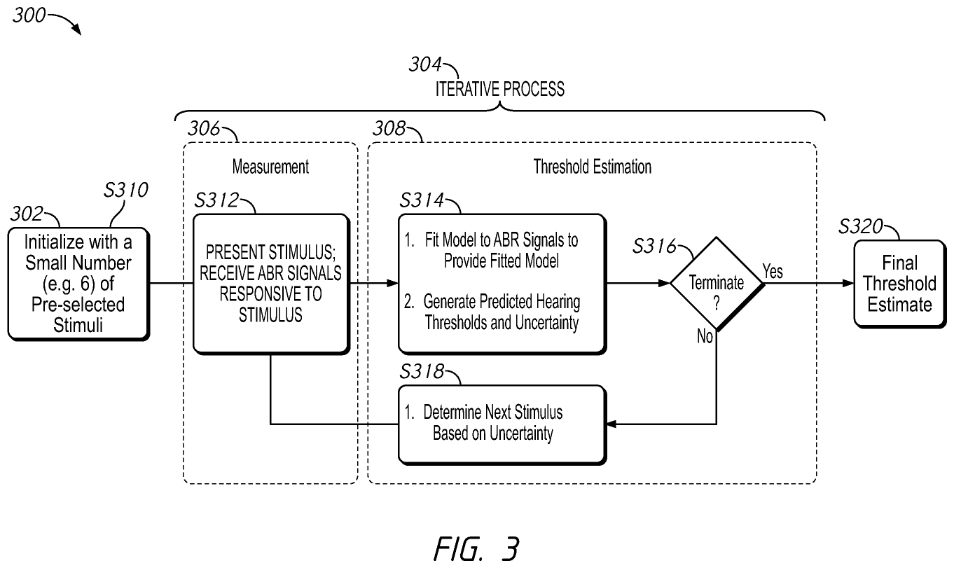

FIG. 3 is a schematic illustration of a flowchart 300 of estimating hearing thresholds using ABR signals in accordance with examples described herein. The flowchart 300 includes an initialization block 302, an iterative process 304, and a final threshold block S320. The iterative process 304 may include a measurement block 306 and a threshold estimation block 308. The actions shown in flowchart 300 of FIG. 3 may be taken by the system 100 of FIG. 1 in some examples, and/or the device 202 of FIG. 2, for example. For example, the executable instructions shown and described in FIG. 1 and/or FIG. 2 may include instructions for performing the operations shown and described in the flowchart 300.

The flowchart 300 is exemplary only. Additional, fewer, and/or different operations may be included in other examples.

While examples are described herein generally relating to a full ABR waveform across frequencies of interest, and the estimation of hearing thresholds, in some examples a particular portion of the ABR waveform may be monitored in accordance with techniques described herein. For example, different portions of the ABR waveform may be associated with particular synapses. A portion of the ABR waveform associated with a particular synapse may be present at a particular latency time from the stimulus time. Accordingly, in some examples, a particular portion of the ABR waveform may be analyzed in accordance with techniques described herein and may result in an estimated threshold and/or performance relating to a particular synapse.

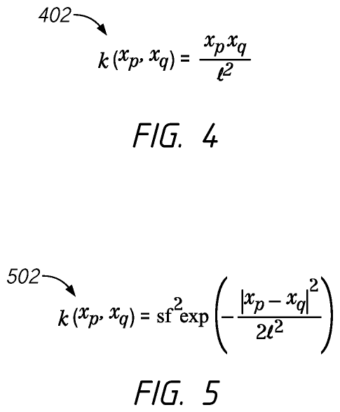

Example operations of the system 100 for estimating hearing thresholds using a fitted model based on ABR signals is described to support the functionality and relevant design decisions are described herein. Example operations of estimating hearing thresholds may be an adaptive procedure that leverages a statistical model to predict the ABR signals, such as auditory evoked potentials, over a wide range of frequency and level combinations. During the procedure, the statistical model is fitted to the ABR signals collected for previously tested stimulus conditions. The fitted statistical model is then used to select the next stimulus to reduce the expected uncertainty of the model predicted ABR threshold across frequencies. In some examples, the statistical model may be a GP model using the GP for machine learning. The GP model may account for covariance between data points in a stimulus space. The GP model may not impose strong a priori assumptions about waveform morphology of the ABR signals. In some examples, the GP model may assume the covariance across three dimensions: time, frequency, and stimulus sound pressure level, which may be fitted simultaneously by the GP model. The covariance between pairs of variables may be calculated based on one or more kernels to form complex composite kernels based on representation of the model. For example, such kernels may include a linear kernel with isotropic distance measure and a squared exponential with isotropic distance measure. FIG. 4 shows an equation 402 representing a linear kernel of a GP model in accordance with examples described herein. FIG. 5 shows an equation 502 representing a squared exponential kernel of a GP model in accordance with examples described herein. In the equations 402 and 502, [symbol] is a characteristic length scale. In the equation 502, sf is a signal variance. The linear kernel represented by the equation 402 may have one hyperparameter, hyp=[log ( [symbol] )]. The squared exponential kernel represented by the equation 502 may have two hyper parameters, hyp=[log ( [symbol] ); log (sf)]. The kernels may be chosen based on a priori expectations of the data covariance across the dimensions of time, frequency, and sound pressure level. The squared exponential kernel may be used for the time dimension because data closer together in time may be assumed to covary more than data that are far apart. Similarly, the squared exponential kernel may be chosen for the base-two logarithm of the frequency dimension, assuming that nearby frequency bands should covary more than frequency bands that are far apart. The covariance kernel characterizing the sound pressure levels of the stimuli may be a product of the squared exponential kernel and linear kernel. While linear amplitude growth with sound pressure levels of the stimuli is a reasonable characterization of ABR signals above threshold, the amplitude growth may deviate from linearity when there is no signal present below threshold. A composite kernel combining the squared exponential and linear kernels may provide a GP model with predicted ABR signals having an amplitude which may drop to zero for stimuli below threshold. The GP model may be fitted across the three dimensions simultaneously, thus a composite kernel may be defined as the product of the kernels applied to each of the three dimensions in the variable space. The composite covariance kernel may include seven hyperparameters: the length scale log ( [symbol] ) and signal variance log (sf) in the time, frequency, and sound pressure level dimensions. The length scale log and the signal variance of the sound pressure level dimension may have an additional length scale (log ( [symbol] )) associated with the linear kernel. The hyperparameters may initially be randomly drawn from wide search ranges, and then tuned via the training. In some examples, search ranges may be selected generally to work for a large number of animal species. In some examples, search ranges may be specifically tailored for a given species. In some examples, estimating the ABR signals across a desired range of frequencies and sound pressure levels may be performed using the stimuli used to train the model as well as stimuli that have not been included. The model of estimated ABR signals may include both the mean and variance of the time series. The variance of amplitude of the ABR signals may be interpreted as the uncertainty in the model for a given time, frequency, and level.

The adaptive procedure of estimating hearing thresholds may include an initializing process 302 and an iterative process 304. The initializing process 302 may use an initial set of stimuli under pre-selected test conditions. The initial set of stimuli are chosen at predominantly high levels in order to ensure that the GP model can correctly characterize the waveform morphology and dependence on level of the peak-to-peak amplitude.

In some examples, the initializing process 302 may include operation S310 using example systems or devices, such as the system 100 described herein. In operation S310, the initial set of stimuli may be presented. In some examples, the number of the initial set of stimuli may be small, such as about five to seven. In some examples, the system 100 may cause the ABR testing device 122 to present the stimuli. For example, the computing device 102 may provide information about the initial set of stimuli to the ABR testing device 122 through the communication interface 140, and the stimuli presentation circuitry 124 of the ABR testing device 122 may present the initial set of stimuli by reproducing a number of audio signals 126 corresponding to the initial set of stimuli through the sound conduction device 132, such as air conductive devices, such as one or more earphones or headphones to be fitted in or outside the one or more corresponding ears of the subject 130 or speakers in proximity to the subject 130, or bone conductive devices, such as one or more speakers, headphones or vibrators to be attached to the head of the subject 130. Furthermore, in operation S310, ABR signals may be received responsive to the initial set of stimuli that have been reproduced. In some examples, the data acquisition circuitry 128 of the ABR testing device 122 may provide the corresponding ABR signals. In some examples, the ABR signals may be auditory evoked potentials in waveforms for one or more corresponding synapses that may occur at different latencies. In some examples, the data acquisition circuitry 128 may obtain at least one auditory evoked potential extracted from ongoing electrical activity of the subject 130 by a sensor 134 of the one or more sensors 134. The ABR testing device 122 may provide the ABR signals to the computing device 102. The communication interface 140 of the computing device 102 may receive the ABR signals at the communication interface 140, and store the ABR signals in the data memory 108. In operation S310, a model may be further fitted to at least the received ABR signals to provide a fitted model. In some examples, the model may be the GP model. In some examples, the fitted model may be used to predict ABR signals as a function of time, frequency and sound pressure level of stimuli. In some examples, in order to fit the model, covariance between pairs of variables may be calculated. In some examples, the calculation may include calculation of covariance between pairs of variables across sound pressure levels using a linear kernel. In some examples, the calculation may include calculation of covariance between pairs of variables across time using a squared exponential kernel. In some examples, the calculation may include calculation of covariance between pairs of variables across base-two logarithm of frequency using a squared exponential kernel. In some examples, the calculation may include calculation of covariance between pairs of variables across sound pressure levels using a covariance kernel that is a product of a squared exponential kernel and a linear kernel. In some examples, using the fitted model, predicted hearing thresholds 118 across a range of frequencies and uncertainty associated with the hearing thresholds may be generated. In some examples, peak-to-peak amplitudes of the predicted ABR signals that have been generated using the fitted model may be calculated. A hearing threshold at each frequency may be defined as a lowest sound pressure level of a stimulus for which the predicted peak-to-peak amplitude of the ABR signals exceeds a predetermined tolerance. The tolerance may be set as a constant or a set based on the statistics that represents a relative strength of the brainstem response to a measurement noise. In some examples, amplitudes of ABR signals responsive to stimuli across the range of frequencies may be predicted using the fitted model, the predicted amplitudes of the ABR signals may be compared with an amplitude of noise of the ABR signals, and predicted hearing thresholds may be generated based on the comparison. For example, predicted hearing thresholds may be generated when the predicted amplitudes of the ABR signals and the amplitude of noise of the ABR signals are substantially the same. In another example, predicted hearing thresholds may be generated when the predicted amplitudes of the ABR signals is a threshold amount higher than the amplitude of noise of the ABR signals.

In some examples, a variance of amplitude of the ABR signals may be estimated, and the uncertainty associated with the hearing thresholds may be based on variance of amplitude of an ABR waveform generated using the fitted model. In operation S310, a next stimulus to be presented to the subject 130 may be determined based on the estimated hearing thresholds. In some examples, the next stimulus may be determined based, at least in part, on the uncertainty associated with the hearing thresholds. In some examples, at least one of a frequency or a sound pressure level of the next stimulus may be selected to reduce uncertainty associated with the hearing thresholds. Thus, the next stimulus may be selected for presenting to the subject 130.

Once the ABR signals under the initial pre-selected test conditions are obtained and the model is initialized, an iterative process 304 may be performed by the system 100. In some examples, the iterative process 304 may include a measurement block 306 and a threshold estimation block 308. For example, the measurement block 306 may be performed by the ABR testing device 122. The threshold estimation block 308 may be performed by the processor 104. The measurement block 306 may include operation S312. In operation S312, the selected stimulus may be presented to a subject 130. For example, the computing device 102 may provide information about the selected stimulus to the ABR testing device 122 through the communication interface 140, and the stimuli presentation circuitry 124 of the ABR testing device 122 may present the initial set of stimuli by reproducing audio signals 126 corresponding to the selected stimulus through the sound conduction device 132, similar to operation S310. Furthermore, in operation S312, one or more ABR signals may be received responsive to the presented stimulus. In some examples, the data acquisition circuitry 128 of the ABR testing device 122 may provide the corresponding one or more ABR signals. In some examples, the ABR signals may be one or more auditory evoked potentials in waveforms for one or more corresponding synapses that may occur at different latencies that may be extracted from ongoing electrical activity of the subject 130 by one or more sensors 134 corresponding to the synapses. The ABR testing device 122 may provide the one or more ABR signals to the computing device 102. The communication interface 140 of the computing device 102 may receive the one or more ABR signals at the communication interface 140, and store the one or more ABR signals in the data memory 108.

For example, the threshold estimation block 308 may be performed by the processor 104. The threshold estimation block 308 may include steps S314, S316 and S318. In operation S314, the model may further be fitted to at least the received ABR signals to provide a fitted model. In some examples, the fitted model may be the GP model. Similarly to the initializing process 302, the fitted model may be used to predict ABR signals as a function of time, frequency and sound pressure level of stimuli. In some examples, in order to fit the model, covariance between pairs of variables may be calculated based on the collected ABR signals. The collected ABR signals may include the ABR signals responsive to the selected stimulus. In some examples, the collected ABR signals may further include ABR signals collected prior to the presentation of the selected stimulus. In some examples, the calculation may include at least one of calculation of covariance between pairs of variables across sound pressure levels using a linear kernel, calculation of covariance between pairs of variables across time using a squared exponential kernel, calculation of covariance between pairs of variables across base-two logarithm of frequency using a squared exponential kernel, or calculation of covariance between pairs of variables across sound pressure levels using a covariance kernel that is a product of a squared exponential kernel and a linear kernel. In some examples, predicted hearing thresholds 118 across a range of frequencies and uncertainty associated with the hearing thresholds may be generated using the fitted model responsive, at least, to the selected stimuli. In some examples, peak-to-peak amplitudes of ABR signals responsive to stimuli across the range of frequencies may be predicted using the fitted model, the predicted amplitudes of the ABR signals may be compared with an amplitude of noise of the ABR signals, and predicted hearing thresholds may be generated based on the comparison. The predicted ABR signals may have a variance associated with their amplitudes. This variance may represent uncertainty in the model, representing generally how well the model predicted waveforms for a given frequency and level can be trusted. In operation S316, the processor 104 may determine if one or more termination criteria is met. If none of the termination criteria is met, the system 100 may transition to operation S318. In operation S318, the processor 104 may determine the next stimulus based, at least in part, on the uncertainty associated with the hearing thresholds at each iteration. In some examples, at least one of a frequency or a sound pressure level to reduce uncertainty associated with the predicted hearing thresholds using the fitted model may be determined. For example, there may be possible schemes for selecting the next stimulus, and one of the four schemes may be selected randomly at each iteration. In some examples, each scheme may be used a particular percentage of iterations. In some examples, each scheme may follow another in a particular order. Additional and/or fewer schemes may be used in other examples.

Scheme 1: The next stimulus is chosen at the sound pressure level and frequency along the estimated threshold contour that meets an uncertainty criterion (e.g., where the predicted ABR waveform has the largest variance). This may be interpreted as sampling at the point along the estimated threshold contour for which the GP model is the least confident.

Scheme 2: At the frequency with an uncertainty along the predicted threshold meeting a particular criterion (e.g., largest variance of amplitude of the ABR waveform) but at a higher level (e.g., a randomly selected higher level). In some examples, the higher level may be below an upper bound imposed by hardware or safety limitations. This scheme may be introduced to help ensure that the selected stimulus may occasionally sample over the dynamic range of the stimulus conditions.

Scheme 3: At the frequency with the uncertainty along the predicted threshold meeting a particular criterion (e.g., largest variance of amplitude of the ABR waveform) but at a lower level. The lower level may be randomly selected in some examples. In some examples, the lower level may be above a lower bound imposed by hardware limitations or a priori knowledge of the threshold. This scheme may be introduced to help ensure that the adaptive stimulus choice will occasionally sample over the dynamic range of the stimulus conditions.

Scheme 4: One or another number of levels below the currently lowest level at the frequency for which the fewest stimuli have been included. In pilot studies, it was observed that frequencies with the fewest stimuli often had predicted thresholds that were higher than expected. This scheme may be introduced to at least partially address that problem in some examples by selecting the frequency with the least data, and sampling below the predicted threshold.

After determining the next stimulus, the system 100 may transition to operation S312 to continue the iterative process 304. If the one or more termination criteria is met, the iterative process 304 may be terminated and the system 100 may perform operation S320, provide the final threshold estimate.

### Implemented Example I

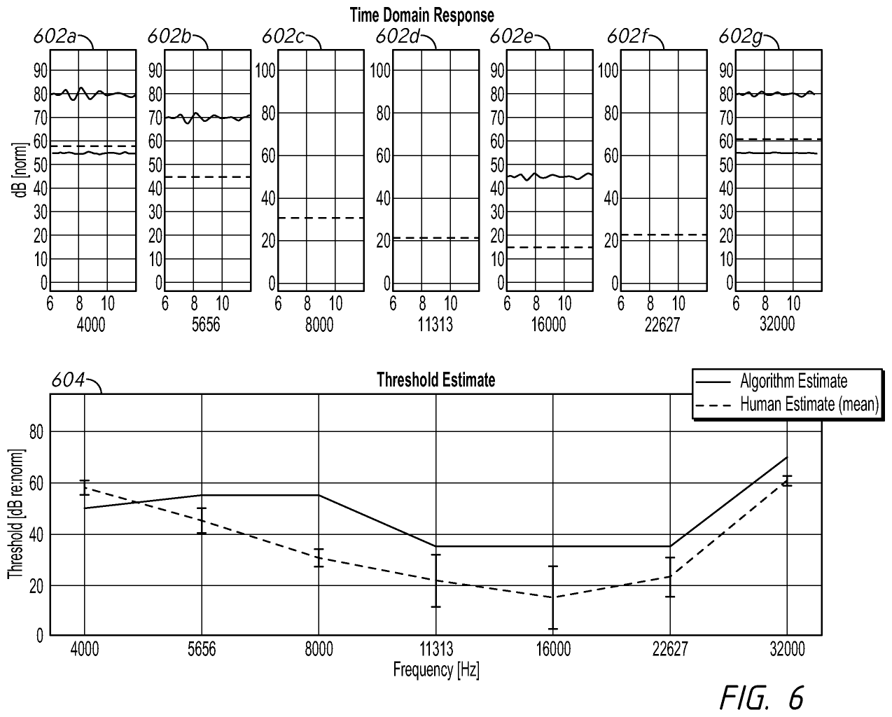

FIGS. 6-9 are diagrams of ABR signals and threshold estimates based on a set of stimuli in accordance with examples described herein. FIGS. 6-9 may be obtained through the adaptive procedure described with the flowchart of FIG. 3, and may utilize the systems shown in FIG. 1 and/or FIG. 2. In FIG. 6, the set of stimuli may be an initial set of stimuli under pre-selected test conditions. Upper diagrams 602a-602g of FIG. 6 show ABR signals responsive to a set of stimuli having given combinations of frequencies and sound pressure levels that is used to fit the GP model. The number of stimuli in the set of stimuli in FIG. 6 is six. The diagrams 602a-602g have horizontal axes representing time (in seconds) and vertical axes representing the stimuli (in dB). For example, the diagram 602a shows two ABR signals responsive to two stimuli in the initial set of stimuli in solid lines, having the levels of approximately 56 dB and 80 dB and a mean human rated threshold in a dotted line, having the level of approximately 58 dB at 4000 Hz. The diagram 602b shows an ABR signal responsive to a stimulus in the initial set of stimuli in a solid line, having the level of approximately 70 dB and a human rated threshold in a dotted line, having the level of approximately 45 dB at 5656 Hz. The diagram 602c shows a human rated threshold in a dotted line, having the level of approximately 30 dB at 8000 Hz. The diagram 602d shows a human rated threshold in a dotted line, having the level of approximately 20 dB at 11313 Hz. The diagram 602e shows an ABR signal responsive to a stimulus in the initial set of stimuli in a solid line, having the level of approximately 45 dB and a human rated threshold in a dotted line, having the level of approximately 16 dB at 16000 Hz. The diagram 602f shows a human rated threshold in a dotted line, having the level of approximately 22 dB at 22627 Hz. The diagram 602g shows two ABR signals responsive to two stimuli in the initial set of stimuli in solid lines, having the levels of approximately 55 dB and 80 dB and a human rated threshold in a dotted line, having the level of approximately 60 dB at 32000 Hz. The lower diagram 604 shows predicted hearing thresholds for a range of frequencies based on the fitted GP model. The diagram 604 has a horizontal axis representing frequency (in Hz) and a vertical axis representing predicted hearing threshold sound pressure level (in dB). A solid line includes the predicted thresholds based on the model and a dotted line shows a mean and standard deviation of hearing thresholds estimated by human raters for comparison. A relatively large difference may be observed between the human rated threshold and the model estimated threshold at 8000 Hz.

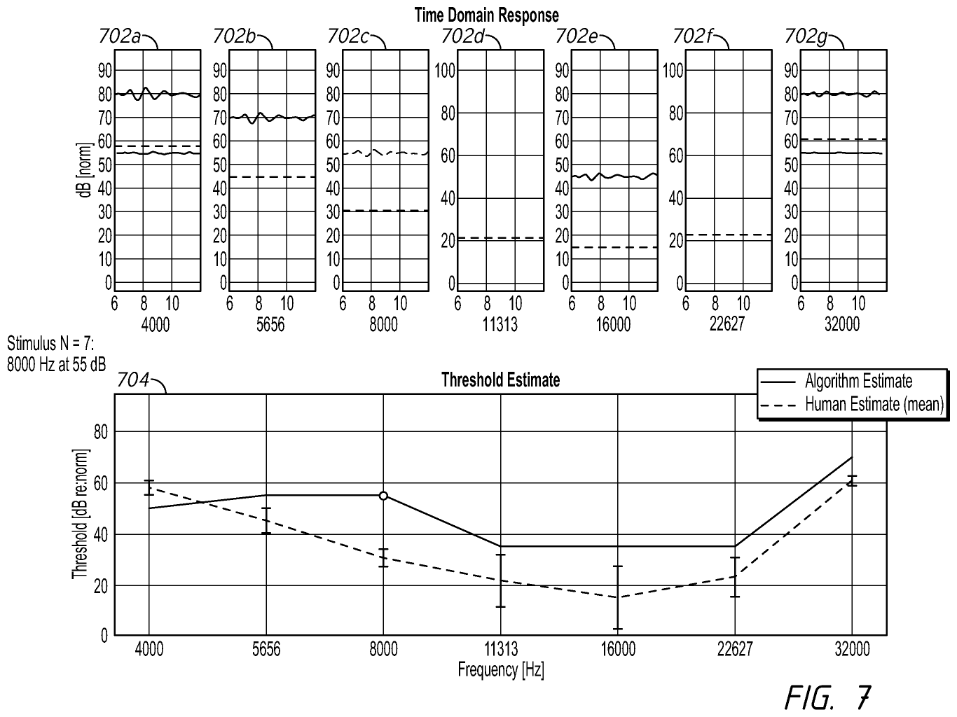

In FIG. 7, the set of stimuli and a next stimulus may be used for model fitting. Upper diagrams 702a-702g of FIG. 7 show ABR signals responsive to the set of stimuli and the next stimulus having given combinations of frequencies and sound pressure levels that is used to fit the GP model. The number of stimuli in FIG. 7 is seven. The diagrams 702a-702g have horizontal axes representing time (in seconds) and vertical axes representing the stimuli (in dB). For example, the diagrams 702a, 702b, 702d to 702g may be similar to the diagrams 602a, 602b, 602d to 602g and the description of these diagrams is omitted for brevity. The next stimulus may be selected based on predetermined schemes, such as one of scheme 1-4. In some examples, the next stimulus may be related to the stimulus condition along the threshold for which the predicted waveform has the highest variance. The diagram 702c shows the human rated threshold in a dotted line for comparison purposes, having the level of approximately 30 dB at 8000 Hz and an ABR signal in a solid line responsive to the next stimulus having the level of approximately 55 dB. The diagram 704 has a horizontal axis representing frequency (in Hz) and a vertical axis representing predicted hearing threshold sound pressure level (in dB). A solid line includes the predicted thresholds based on the model and a dotted line shows a mean and standard deviation of hearing thresholds estimated by human raters for comparison. Based on the next stimulus, the model may be further fitted as shown in the diagram 704. Generally, examples of systems described herein may create estimates of hearing thresholds that agree well with hearting thresholds calculated using more time-consuming techniques, such as presenting larger numbers of stimuli to a subject in an exhaustive fashion (e.g., without choosing next stimuli based on predicted ABR signals as described herein.)

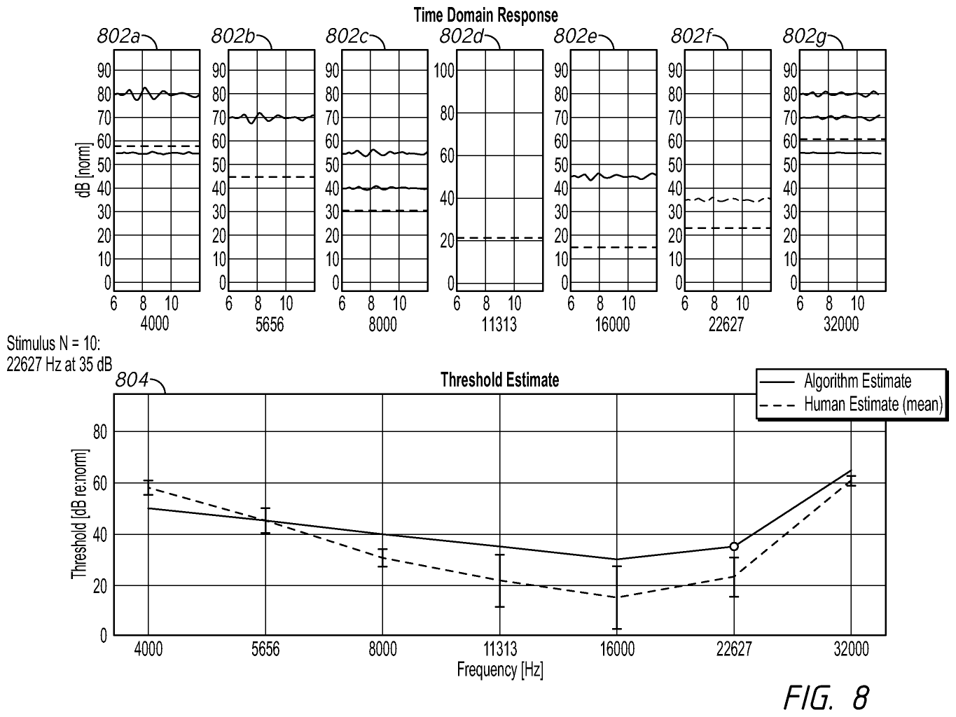

In FIG. 8, the set of stimuli and additional stimuli may be used for model fitting. Upper diagrams 802a-802g of FIG. 8 show ABR signals responsive to the set of stimuli and additional stimuli having given combinations of frequencies and sound pressure levels that is used to fit the GP model. The number of stimuli in FIG. 7 is ten. The diagrams 802a to 802g have horizontal axes representing time (in seconds) and vertical axes representing the stimuli (in dB). For example, the diagrams 802a, 802b, 802d, 802e may be similar to the diagrams 702a, 702b, 702d, 702e and the description of these diagrams is omitted for brevity. The ABR signals responsive to three additional stimuli presented in three iterations having the levels of approximately 40 dB, 35 dB and 70 dB at frequencies of 8000 Hz, 22627 Hz, and 32000 Hz are shown in solid lines in the diagrams 802c, 802f and 802g, respectively. The diagram 804 has a horizontal axis representing frequency (in Hz) and a vertical axis representing predicted hearing threshold sound pressure level (in dB). A solid line includes the predicted thresholds based on the model and a dotted line shows a mean and standard deviation of hearing thresholds estimated by human raters for comparison. Based on the next stimulus, the model may be further fitted as shown in the diagram 804.

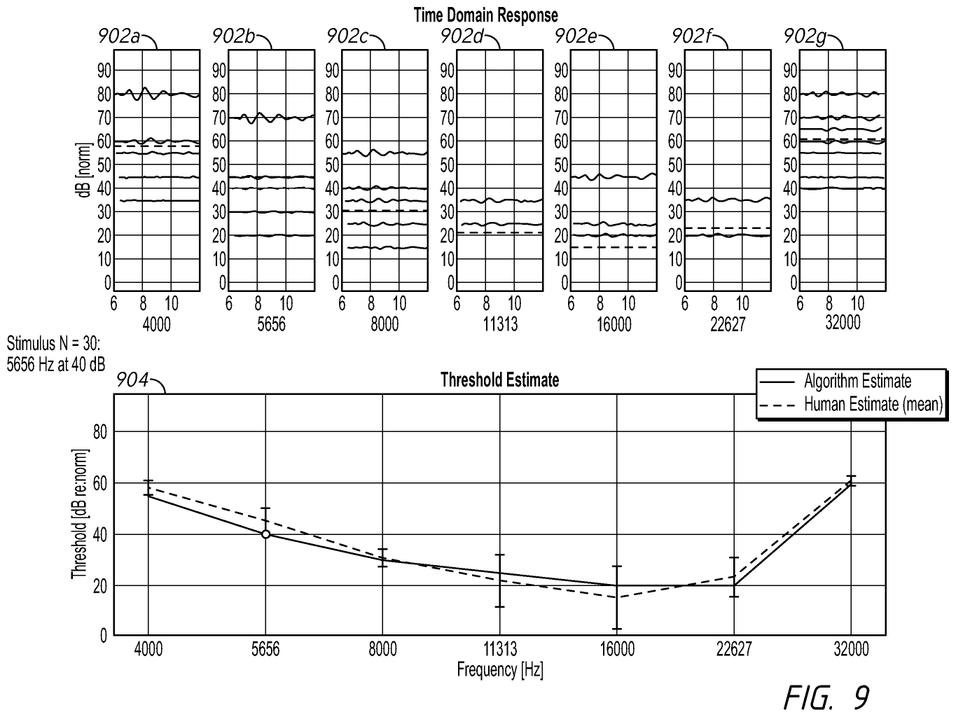

In FIG. 9, the set of stimuli and additional stimuli may be used for model fitting. Upper diagrams 902a-902g of FIG. 9 show the set of stimuli and additional stimuli having given combinations of frequencies and sound pressure levels that is used to fit the GP model. The number of stimuli in FIG. 9 is 30. The diagrams 902a to 902g have horizontal axes representing time (in seconds) and vertical axes representing the stimuli (in dB). The ABR signals of a total of 30 stimuli, including 24 additional stimuli presented in 24 iterations are shown in solid lines in the diagrams 902a to 902g. The diagram 904 has a horizontal axis representing frequency (in Hz) and a vertical axis representing predicted hearing threshold sound pressure level (in dB). A solid line includes the predicted thresholds based on the model and a dotted line shows a mean and standard deviation of hearing thresholds estimated by human raters for comparison. Based on the next stimulus, the model may be further fitted as shown in the diagram 904. By using the model fitting, without providing a large set of stimuli, the model may be fitted to provide predictive hearing thresholds.

### Implemented Example II

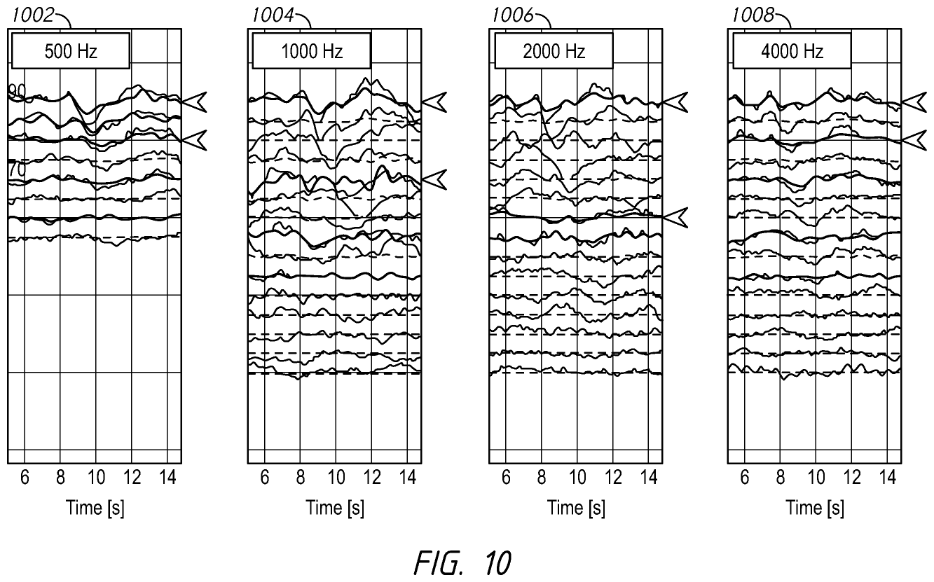

FIG. 10 shows example ABR signals data sets of a system of estimating hearing thresholds using ABR signals in accordance with examples described herein. FIG. 10 was obtained through the adaptive procedure described with the flowchart of FIG. 3. Seven normal hearing adult human subjects were recruited. Stimuli were presented through earphone inserted in ears of the subjects. Sensors, such as electrodes were attached to the subjects. For example, a positive electrode was attached to the high forehead of each subject, a couple of negative electrodes were attached to mastoid bones behind the ears of each subject, and a ground electrode was attached to the low forehead of each subject. Stimuli were prepared to have tone bursts in 5 dB decrements from a 90 dB sound pressure level to below threshold at 0.5, 1.0, 2.0, 4.0 kHz. The grand mean of stimuli for each subject included repetitions after artifact rejection. The datasets were collected across two 2-hour ABR sessions. ABR signals from three of the seven subjects contained substantial artifacts, likely electrical noise, and were excluded from the analysis.

FIG. 10 includes dataset diagrams 1002, 1004, 1006 and 1008, showing ABR signals responsive to a set of stimuli having given combinations of frequencies and sound pressure levels that is used to fit the GP model, and demonstrating a fitted model. The dataset diagrams 1002, 1004, 1006 and 1008 each correspond to a particular frequency. The dataset diagrams 1002, 1004, 1006, and 1008 each have horizontal axes representing time (in seconds) and vertical axes representing ABR signals responsive to the stimuli (in dB). For example, the dataset diagrams 1002, 1004, 1006 and 1008 include thin lines with data, thick black lines representing predicted ABR signals over time responsive to stimuli at particular levels and frequencies, thick black lines with arrows representing ABR signals responsive to an initial set of stimuli in the stimuli, and thick grey lines representing ABR signals of a predicted model. For example, the dataset diagrams 1002, 1004, 1006 and 1008 show the data obtained at 500 Hz, 1000 Hz, 2000 Hz, and 4000 Hz, respectively. Systems described herein, such as the system of FIG. 1 and/or FIG. 2 may be used to generate the data shown in the diagrams of FIG. 10.

As described herein, a parameter space of ABR signals, both received and predicted, may be generated across time, frequency, and stimulus level. As shown in FIG. 10, approximately 15 milliseconds of ABR signal amplitudes are collected or predicted for each frequency and stimulus level. As shown in FIG. 10, four frequencies are used. Any number of frequencies may be used in other examples, generally ranging across audible frequency ranges. In the example of FIG. 10, sound pressure levels of 20 to 90 dB may be used, although other ranges may be used in other examples. In the example of FIG. 10, ABR signal amplitudes may be received and/or predicted corresponding to stimulus levels 5 dB apart—e.g., at 20, 25, 30, 35, 45, 50, 55, 60, 65, 70, 75, 80, 85, and 90 dB. Other intervals may be used in other examples. In some examples, ABR signals at each of these stimulus levels may be received and/or predicted at each frequency used. In other examples, not all sound levels may be used for ABR signal generation at each frequency level. In this manner, some ABR signals may be received responsive to stimuli, and other ABR signals may be predicted by fitting a model to the received data. Fitting the model over frequency, time, and level, results in a representative modeled parameter space. The parameter space can be used to estimate hearing thresholds at particular frequencies and/or continuously across a range of frequencies. At least in part because there may be variances associated with the amplitudes of the predicted ABR signals, the hearing thresholds may have uncertainty associated with them. As described herein, systems may select one or more next stimuli to present to a subject based on the uncertainty such that, after receipt of additional ABR signals from the subject, the uncertainty in the estimated hearing thresholds may be reduced.

Threshold estimates responsive to the example ABR signals were obtained through the adaptive procedure described with the flowchart of FIG. 3. The predicted thresholds based on the model and mean and standard deviation of hearing thresholds estimated by human raters were obtained for comparison between estimated thresholds using fitted model and rater estimated thresholds for different subjects. For each subject, the threshold is estimated by four raters and four runs of the algorithm.

The fitted model and rater estimated thresholds match reasonably well when fewer stimuli were used. The rest/retest reliability of using the fitted model is better than inter-rater reliability. Considerable inter-rater differences were likely due to the noisy data. Algorithm performance using the fitted model on the noisy data suggests that the statistical criterion for threshold may improve the robustness to the noise. Standard deviations of estimates using the fitted model from successive runs are generally smaller than standard deviations of inter-rater estimates. Thus, stimuli presentation with prediction of hearing thresholds using the fitted model may be terminated earlier than stimuli presentation with rating of a fixed set of stimuli by human raters without a strong impact on estimated threshold.

From the foregoing it will be appreciated that, although specific embodiments of the disclosure have been described herein for purposes of illustration, various modifications may be made without deviating from the spirit and scope of the disclosure.

The particulars shown herein are by way of example and for purposes of illustrative discussion of the preferred embodiments of the present disclosure.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments, or processes described herein may be combined with one or more other examples, embodiments, and/or processes or be separated and/or performed among separate devices or device portions in accordance with the present systems, devices, and methods.

Finally, the above discussion is intended to be merely illustrative of the present method, system, device and computer readable medium and should not be construed as limiting the appended claims to any particular embodiment or group of embodiments. Thus, while the present method, system, device and computer readable medium have been described in particular detail with reference to exemplary embodiments, it should also be appreciated that numerous modifications and alternative embodiments may be devised by those having ordinary skill in the art without departing from the broader and intended spirit and scope of the present method, system, device and computer readable medium as set forth in the claims that follow. Accordingly, the specification and drawings are to be regarded in an illustrative manner and are not intended to limit the scope of the appended claims.

What is claimed is:

1. A method comprising:
presenting at least one stimulus to a subject;
receiving first auditory brainstem response (ABR) signals responsive to the at least one stimulus;
fitting a model to at least the first ABR signals to provide a fitted model;
generating, using the fitted model, predicted hearing thresholds across a range of frequencies and uncertainty associated with the predicted hearing thresholds;
determining a next stimulus based, at least in part, on the uncertainty associated with the hearing thresholds; and
presenting the next stimulus to the subject.

2. The method of claim 1, further comprising performing at least one iterative operation, the iterative operation including:
receiving a second ABR signal responsive to the next stimulus;
fitting the previously fitted model to at least the second ABR signal to provide a second fitted model;
generating, using the second fitted model, second predicted hearing thresholds across a range of frequencies and uncertainty associated with the second hearing thresholds;
determining a second next stimulus based, at least in part, on the uncertainty associated with the second hearing thresholds; and
presenting the second next stimulus to the subject.

3. The method of claim 1, wherein presenting the at least one stimulus comprises providing at least one audio signal corresponding to the at least one stimulus to at least one sound reproducing device in proximity to the subject; and
wherein the first ABR signals comprise voltages provided by a sensor on the subject.

4. The method of claim 1, wherein generating the first predicted hearing thresholds comprises:
predicting amplitudes of ABR signals responsive to stimuli across the range of frequencies and sound pressure levels using the fitted model;
comparing the predicted amplitudes of the ABR signals with an amplitude of noise of the ABR signals; and
generating the predicted hearing thresholds based on the comparison.

5. The method of claim 4, further comprising calculating at least one of peak-to-peak amplitudes of the predicted ABR signals, amplitudes of individual peaks in the predicted ABR signals or latencies of individual peaks in the predicted ABR signals generated using the fitted model.

6. The method of claim 4, wherein the fitted model is used to predict ABR signals as a function of time, frequency and sound pressure level of the stimuli.

7. The method of claim 6, wherein the model is a Gaussian Process model.

8. The method of claim 6, wherein fitting the model comprises calculating covariance between pairs of variables across sound pressure levels using a linear kernel.

9. The method of claim 6, wherein fitting the model comprises calculating covariance between pairs of variables across time using a squared exponential kernel.

10. The method of claim 6, wherein fitting the model comprises calculating covariance between pairs of variables across base-two logarithm of frequency using a squared exponential kernel.

11. The method of claim 6, wherein fitting the model comprises calculating covariance between pairs of variables across levels using a covariance kernel that is a product of a squared exponential kernel and a linear kernel.

12. The method of claim 6, wherein determining the next stimulus comprises selecting at least one of a frequency or a sound pressure level to reduce uncertainty associated with the hearing thresholds.

13. The method of claim 12, wherein the uncertainty associated with the hearing thresholds is based on variance of amplitude of an ABR waveform generated using the fitted model.

14. A system comprising:
an ABR testing device; and
a computer coupled to the ABR testing device comprising:
a processor; and
a non-transitory computer readable medium storing computer-executable instructions which, when executed, cause the processor to perform operations comprising:
causing the ABR testing device to present at least one stimulus to a subject;
receiving first ABR signals from the ABR testing device responsive to the at least one stimulus;
fitting a model to at least the first ABR signals to provide a fitted model;
generating, using the fitted model, predicted hearing thresholds across a range of frequencies and uncertainty associated with the predicted hearing thresholds;
determining a next stimulus based, at least in part, on the uncertainty associated with the hearing thresholds; and
presenting the next stimulus to the subject.

15. The system of claim 14, wherein the ABR testing device comprises the computer.

16. The system of claim 14, wherein the ABR testing device comprises stimuli presentation circuitry configured to provide at least one audio signal corresponding to the at least one stimulus to a sound conduction device on the subject that is coupled to the ABR testing device.

17. The system of claim 14, wherein the ABR testing device further comprises data acquisition circuitry coupled to at least one sensor in proximity to the subject,
wherein the data acquisition circuitry is configured to provide the first ABR signals responsive to at least one auditory evoked potential extracted from ongoing electrical activity of the subject by the sensor.

18. The system of claim 17, wherein the at least one sensor is an electrode on the subject, the at least one sensor configured to generate the first ABR signals responsive to the at least one auditory evoked potential.

19. A non-transitory computer readable medium encoded with instructions which, when executed, cause a system to perform operations comprising:
presenting at least one stimulus to a subject;
receiving first ABR signals responsive to the at least one stimulus;
fitting a model to at least the first ABR signals to provide a fitted model;
generating, using the fitted model, predicted hearing thresholds across a range of frequencies and uncertainty associated with the predicted hearing thresholds;
determining a next stimulus based, at least in part, on the uncertainty associated with the hearing thresholds; and
presenting the next stimulus to the subject.

20. The non-transitory computer readable medium of claim 19, wherein the operations further comprises performing at least one iterative operation comprising:
receiving a second ABR signal responsive to the next stimulus;
fitting the previously fitted model to at least the second ABR signal to provide a second fitted model;
generating, using the second fitted model, second predicted hearing thresholds across the range of frequencies and uncertainty associated with the second hearing thresholds;
determining a second next stimulus based, at least in part, on the uncertainty associated with the second hearing thresholds; and
presenting the second next stimulus to the subject.

21. The non-transitory computer readable medium of claim 19, wherein generating the first predicted hearing thresholds comprises:
predicting amplitudes of ABR signals responsive to stimuli across a range of frequencies and sound pressure levels using the fitted model;
comparing the predicted amplitudes of the ABR signals with an amplitude of noise of the ABR signals; and
generating the first predicted hearing thresholds based on the comparison.

22. The non-transitory computer readable medium of claim 19, wherein determining the next stimulus comprises selecting at least one of a frequency or a sound pressure level to reduce the uncertainty associated with the hearing thresholds.

23. The non-transitory computer readable medium of claim 19, wherein the uncertainty associated with the hearing thresholds is based on variance of amplitude of an ABR waveform generated using the fitted model.
